(19) United States
(12) Patent Application Publication
(10) Pub. No.: US 2007/0060891 A1
(43) Pub. Date: Mar. 15, 2007

(54) Implant with a skin penetration section

(76) Inventors: Richard Skiera, Wien (DE); Michael Meyer, Falkensee (DE); Marc Kraft, Birkenstein (DE)

Correspondence Address: FAEGRE & BENSON LLP;PATENT DOCKETING, 2200 WELLS FARGO CENTER, 90 SOUTH SEVENTH STREET, MINNEAPOLIS, MN, 55402-3901, US

(21) Appl. No.: 10/582,152

(22) PCT Filed: Nov. 25, 2004
(86) PCT No.: PCT/DE04/02628

§ 371 Date: Jun. 8, 2006

(30) Foreign Application Priority Data
Dec. 8, 2003 (DE) 103 57 579.0

(51) Int. Cl. A61M 5/32

(52) U.S. Cl. 604/175

(57) ABSTRACT

An implant including an implant body configured for retention within a body comprising an implant section, a skin penetration section, and extracorporeal connector section, and a planar part surrounding and adjoining the skin penetration section in a sterile connection. The planar part is configured for adhering to skin tissue, and includes a surface configured for supporting a skin layer surrounding the skin penetration section.

The invention relates to an implant with an implant body, comprising an implant section intended to remain in the body, a skin penetration section, and an extracorporeal connector section, and with a planar part which surrounds the skin penetration section, is provided for adhering to skin tissue and has a surface forming a support for a skin layer surrounding the skin penetration section.

Different types of implants are known that are implanted in the human or animal body permanently, i.e. for several months or years, in order to permanently establish a required connection to the interior of the body.

EP 0 867 197 discloses an implant of this kind in the form of a port element through which medication can be passed into the interior of the body via a catheter placed in the interior of the body. The port element forming the implant body is connected to the catheter in the interior of the body and protrudes outward through a skin passage where it forms a connector section that can be covered by a cap and via which the medication can be introduced into the interior of the body, for example with the aid of a syringe.

WO 01/97718 A1 discloses an implant of this kind which is intended for securing a prosthesis to an amputation stump. In this case, the implant body is designed as a load-transmitting part and is connected to a bone in the amputation stump via an anchoring section. In this way, a prosthesis is no longer secured in the customary manner via the soft tissue and the skin of the amputation stump, but instead (imitating the attachment of the natural, amputated limb) directly to the bone from which the amputated limb has been separated. In this case too, it is known for the load-bearing part in the skin penetration section to be equipped with a planar part provided with through-openings into which tissue below the skin passage is intended to grow so that the planar part becomes incorporated in the tissue.

EP 0 367 354 discloses an implant of the type mentioned at the outset, in which the planar part is composed of a metal fabric structure, preferably of titanium or a titanium alloy. The structure is highly porous and is intended to allow skin tissue to grow into and through the planar part. The planar, subcutaneous part of the implant is held by a sleeve into which a skin penetration part can be screwed. The skin penetration part is also sleeve-shaped and protrudes outside of the skin. An implant of this kind is used for conveying medicaments, electrical energy or electrical information to and from an implanted device. The percutaneous implant is positioned via the planar part. Therefore, the planar part with the attached securing sleeve with covered thread is fitted in the skin until the skin has adhered to the planar part. Thereafter, the thread cover is removed and the percutaneous part is screwed in, and the line is fitted into the body.

Particularly in the design of the implant body as a load-bearing part, but also in the case of implanted catheters, the skin penetration area forms a problem zone. As the soft tissue shifts, this causes relative movements between the implant body and the surrounding wound margin at the skin penetration area, as a result of which a clean fusion of the skin to the implant body and to the planar part connected thereto is impeded. A wound that does not heal cleanly, however, represents a constant source of risk of infections and of resulting inflammatory processes. Even the known production of implant bodies from biocompatible material, for example hydroxyapatite or similar materials with which the implant body can also be coated, does not permit the required uniform wound healing if frequent relative movements take place between skin and implant body, as a result of which wounds that have already partially healed may also tear open again.

Therefore, the object of the present invention is to design an implant of the type mentioned at the outset in such a way as to permit improved wound healing in the area of the skin penetration.

According to the invention, in order to achieve this object, an implant of the type mentioned at the outset is characterized in that the planar part adjoins the skin penetration section in a sterile manner and comprises, in one integral piece, an inner area adjoining the skin penetration section, and an outer area, the inner area being made so stiff that it forms an inherently fixed support surface for a skin layer surrounding the skin penetration section, and the outer area has an elasticity adapted to the elasticity of the surrounding tissue.

By means of the elasticity of the planar part in the outer area, the implant according to the invention permits adaptability of the planar part to movements of the skin, so that an effective connection of the planar part to the tissue of the skin can be obtained without strong shearing forces arising between the skin and the planar part, which shearing forces could again destroy the connection between the tissue and the planar part. By contrast, the planar part has a high degree of stiffness in the inner area, that is to say in the area with which the planar part adjoins the skin penetration section of the implant body. This means that the skin adhering to the planar part is by and large not exposed to any movements in the inner area of the planar part, that is to say there are no constantly reopening wound areas. The relative movement between the implant body and the surrounding tissue, including the surrounding skin, is taken up by the elasticity of the planar part, and the connection to the tissue into which the planar part has become incorporated is preserved. The wound margin at the skin penetration site is protected against movements and can heal undisturbed. By means of the sterile connection of the planar part to the skin penetration section, the planar part forms an effective barrier against incoming pathogens, since these can get into the interior of the body on the proximal face of the planar part only via the detour formed by the planar part.

In a preferred embodiment, the elasticity of the planar part increases continuously toward the outside, as a result of which the ability to take up relative movements and stresses increases with the greater available surface area. This effect will be enhanced by the fact that the outer area of the planar part has an elasticity approximating to the elasticity of the skin.

The sterile connection, according to the invention, between the planar part and the implant body can be achieved by exerting an axial pressure, in a manner explained in more detail below, on an annular bearing surface between implant body and planar part. Alternatively, an adhesive join is possible between planar part and implant body. It is also possible for the planar part to be formed in one piece with the implant body. This is especially the case when, for example, the implant body is made of silicone with a high Shore hardness, for example, and the planar part is formed integrally on the implant body, a Shore hardness being obtained which decreases toward the outer area and which alone, or in combination with corresponding configurations of the planar part, produces the desired adaptation of elasticity to the elasticity of the surrounding skin. The sterile connection, together with the planar part, ensures a microbe barrier by which the passage of microbes into the body through the skin penetration site is prevented or made difficult.

The planar part is intended to be made from a bioactive or bioinert material. A suitable material is silicone. It is possible in this case for the planar part to be made in one piece or to be made up of several individual pieces connected to one another in a separable or non-separable manner, in which case the individual pieces can also be made from different materials.

The anchoring of the planar part to the surrounding tissue is promoted, in a manner known per se, by a multiplicity of through-openings through which tissue can grow.

The anchoring to the tissue is promoted by the fact that the planar part is coated with bioactive material, at least on part of its surface, and preferably in an island pattern with interstices. The material preferred for this purpose is hydroxyapatite. This bioactive coating applied in an island pattern affords the advantage that the flexibility or elasticity of the planar part is not impaired by the ceramic-type coating.

The elasticity provided according to the invention is preferably achieved by a suitable shaping of the planar part when the latter is made from a single material, for example silicone. The outwardly increasing elasticity can be obtained by a suitable, preferably continuous reduction of the material thickness, but also by an increasing proportion of a hole surface relative to the material surface. Another embodiment is one in which the material of the planar part, for example silicone, is designed toward the outside with a lower (Shore) hardness. It is of course also possible to modify the material in the outer area of the planar part such that the higher elasticity is obtained in the outer area.

The anchoring of the tissue to the planar part can be further enhanced by at least one surface of the planar part being provided with groove-like depressions or with raised webs. Alternatively, or in addition to this, shaped elements promoting the anchoring of tissue can be applied at least on one part of at least one surface of the planar part. In addition, or alternatively, it is expedient for the surface of the planar part to be designed, at least in some areas, in such a way that adherence of tissue is promoted, for example by means of a porous or roughened structure or by suitable chemical functionalizing of the surface, by which, for example, coupling of proteins is promoted.

By contrast, it may be expedient for the underside of the planar part to be designed such that adherence of tissue is avoided. For this purpose, the underside can be provided with a surface that repels microbes. In this way, so-called biofouling can be avoided.

The planar part preferably has a substantially circular periphery. In this case, the planar part can adjoin the implant body substantially at right angles to a longitudinal axis of the implant body. For adaptation to the soft-tissue contour, particularly in the case of an amputation stump, it can be advantageous if the planar part is shaped like the top of a mushroom, with a jacket surface forming an acute angle with respect to a longitudinal axis of the implant body. For this purpose, the surface can be curved continuously or can also be provided with a stronger curvature and merge into a rectilinear end section.

Particularly if the implant body is anchored in a bone in order to be able to transmit loads, it can be expedient if the planar part is connected releasably to the implant body. In the case of a releasable connection, but also in the case of a non-releasable connection, the stability of the connection of the planar part to the implant body can be improved by the fact that the planar part has a stepped diameter reduction forming an annular shoulder on which the planar part preferably bears. The sterile connection provided according to the invention is then produced by laying the planar part flat on the annular shoulder of the implant body with axial pretensioning.

Alternatively, in the case of a non-releasable connection, an adhesive or weld filling a peripheral gap is provided between the implant body and the planar part.

If, in a preferred embodiment of the invention, the implant body is provided with a bioactive surface at least in the area of connection to the distal surface of the planar part, the skin stabilized by the inner area of the planar part can also grow onto the implant body and thus produce a completely tight connection to the implant body. Growth of the skin higher up on the implant body is avoided by the latter being provided, in its connector section, with a surface that prevents cells from settling on it. This can be effected using a surface made of bioinert or smooth material and by a suitable coating.

The implant according to the invention can, if appropriate, be designed such that several implant bodies are connected to a common planar part, so that the planar part surrounds the several implant bodies and is connected to them in a sterile manner.

Conversely, it is also possible, starting from an implant section, for the implant body to branch into several skin penetration sections which are each connected to a planar part.

The invention will be explained in more detail below on the basis of illustrative embodiments shown in the drawing, in which:

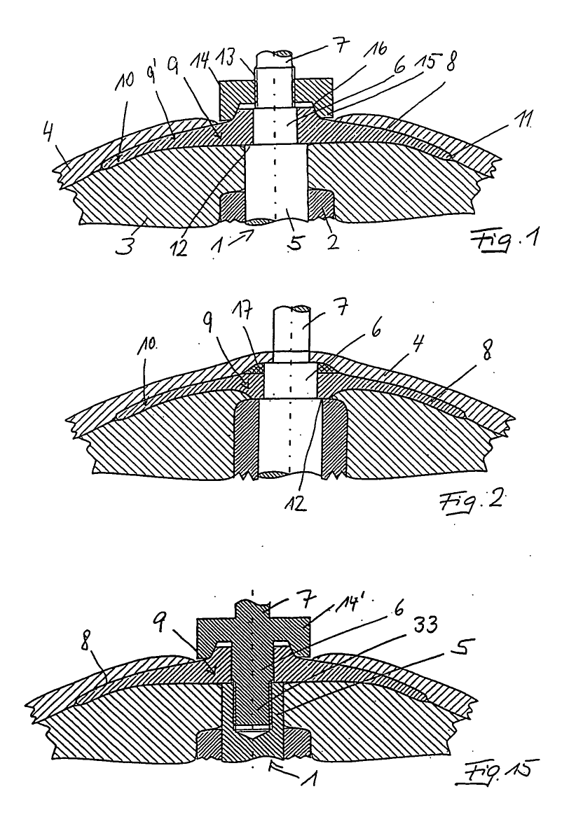

FIG. 1 shows a cross section through a first embodiment of an implant according to the invention after it has been implanted and become incorporated,

FIG. 2 shows a view according to FIG. 1 in a second embodiment,

FIG. 3 shows a schematic and perspective sectional view of a planar part with apertures,

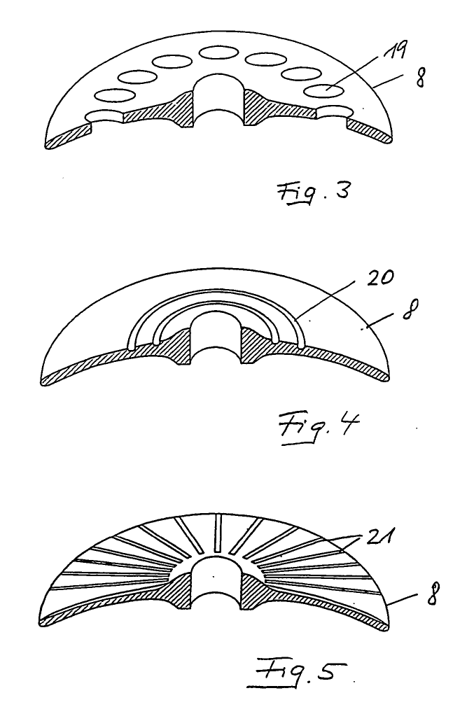

FIG. 4 shows a schematic view, according to FIG. 3, of a planar part with grooves arranged in circles,

FIG. 5 shows a view according to FIG. 4, with radially arranged grooves,

FIG. 6 shows a view, according to FIG. 4, of a planar part on which a porous, sponge-like structure is applied,

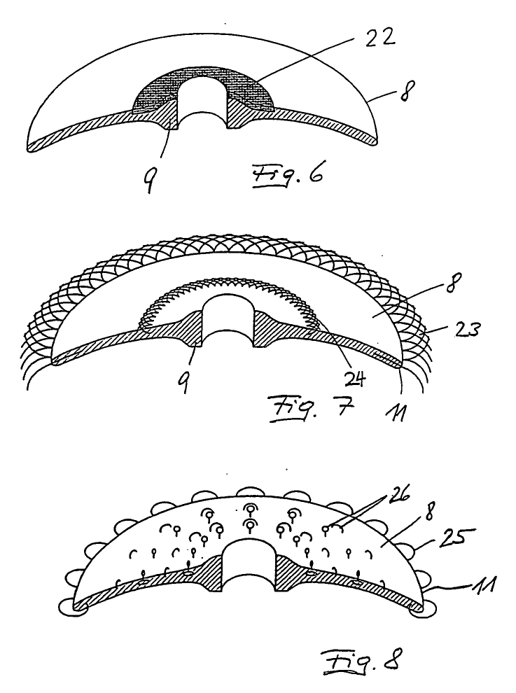

FIG. 7 shows a view, according to FIG. 4, of a planar part with mesh-like structures that protrude from the material of the planar part,

FIG. 8 shows a view, according to FIG. 7, with ring-like and/or eyelet-like shaped elements,

FIG. 9 shows a plan view of a planar part with radially open through-openings in the outer area,

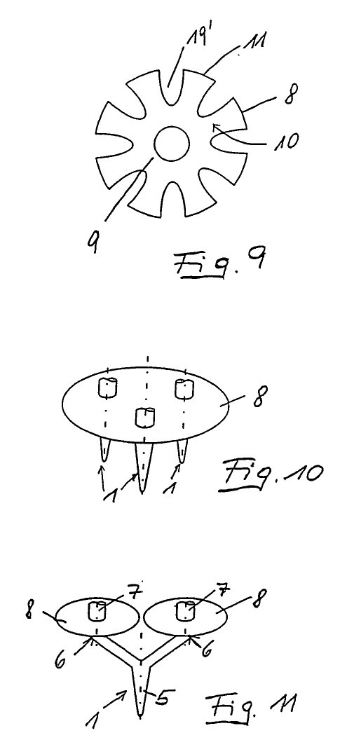

FIG. 10 shows a schematic view of a planar part that surrounds several implant bodies,

FIG. 11 shows a schematic view of an implant body with two skin penetration sections that are each surrounded by a planar part,

FIG. 12 shows a variant design of a surface of the planar part,

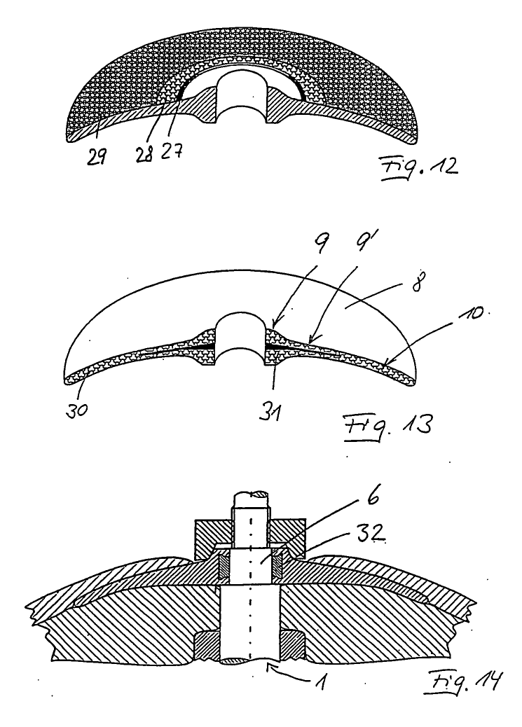

FIG. 13 shows a variant of the structure of the planar part with a mesh-like structure and with a stiffening insert in the inner area and in a transition area,

FIG. 14 shows a variant of the first embodiment according to FIG. 1, with a stiffening insert in the inner area of the planar part,

FIG. 15 shows a further variant of the first embodiment according to FIG. 1, with the implant body being designed in several parts.

FIG. 1 shows an implant body 1 in an amputation stump that includes a tubular bone 2 and a contractile soft tissue 3 which surrounds the tubular bone 2 and is closed distally by a substantially non-contractile skin layer 4. The implant body 1 is inserted into the tubular bone 2 with an implant section 5, which thus serves as an anchoring section. The implant section 5 is adjoined distally by a skin penetration section 6 and, distally from this, by a connector section 7 in the form of a coupling piece for a prosthesis part.

The skin penetration section 6 is connected to a planar part 8 that surrounds the skin penetration section 6. The planar part 8 has an inner area 9 which is designed with a material thickness affording the desired stiffness of the inner area 9. By way of a narrowing of the material that forms a transition area 9′, the inner area 9 merges into an outer section 10 in which the planar part 8 has a considerably smaller material thickness, which decreases further toward the outer edge 11.

The planar part 8 is made of silicone, for example, which is very stiff in the inner area 9 because of the material thickness there. Because of the lesser material thickness in the outer area 10, the planar part 8 has, in the outer area 10, a pronounced elasticity, which can increase toward the outer edge 11 and has the effect that the mobility of the planar part 8 in the outer area 10 corresponds approximately to the mobility of the surrounding skin 3, 4.

It will also be seen from FIG. 1 that the implant body 1 merges from the implant section 5 to the skin penetration section 6 via a stepped diameter reduction, by which means an annular shoulder 12 is formed on which the inner area 9 of the planar part 8 rests. To produce a sterile connection, the inner area 9 is placed on the annular shoulder 12 under axial pretensioning. For this purpose, the connector area 7 adjacent to the skin penetration section 6 is provided with an outer thread 13 onto which a nut 14 is screwed which, with a projecting ring 15, presses on the transition between the inner area 9 and the outer area 10 of the planar part 8. By virtue of a conical profile of a radial inner wall 16 of the ring 15 that interacts with a corresponding conical profile on the distal face of the planar part 8, the axial pretensioning by the nut 14 also leads to a radially inwardly directed pressing force, with which a small gap possibly present between the skin penetration section 6 and the inner area 9 of the planar part 8 is closed, so that in this way a sterile connection is guaranteed between the implant body 1 and the planar part 8.

In the illustrative embodiment shown in FIG. 2, the planar part 8 is connected to the skin penetration section 6 by adhesive that completely fills the gap between skin penetration section 6 and planar part 8. In this illustrative embodiment, the distal face of the inner area 9 is formed by a ring 17 of bioactive material, for example hydroxyapatite.

As will also be seen from the illustrative embodiment according to FIG. 1, the skin penetration section 6 merges into the connector section 7 via a further diameter reduction. If the area of the connector section 7 adjoining the skin penetration section 6 proximally is also coated with a bioactive material, the skin 4 grows onto this area of the connector section 7, as is represented in FIG. 2.

FIG. 3 illustrates how the planar part 8 can be provided with circular through-openings 19 through which tissue 3, 4 can grow in order to anchor the planar part 8 in the skin. The through-openings 19 can also be used for immediate fixing of the planar part 8 by suture connections upon insertion of the implant.

In the illustrative embodiment shown in FIG. 4, the planar part 8 has, in the distal surface, circular grooves 20 into which the skin tissue 4 can grow for anchoring purposes and which ensure an improved supply of blood to the flap of skin lying over them.

In the illustrative embodiment according to FIG. 5, corresponding radial grooves 21 are provided in the distal surface of the planar part 8 for the same purpose.

The effects of improved anchoring and blood supply, that can be achieved using the grooves 20, 21 in the illustrative embodiments shown, can similarly be achieved using webs that protrude from the surface, instead of using the grooves 20, 21.

FIG. 6 shows an illustrative embodiment of a planar part 8 in which the distal face is formed in the inner area 9 by a ring 22 of a sponge-like, porous structure.

Mesh structures 23, 24 protruding from the planar part 8 can also be used for anchoring the tissue 3, 4. In the illustrative embodiment shown in FIG. 7, a mesh structure 23 is provided which protrudes radially from the outer edge 11 of the planar part 8 and which is supplemented by a mesh structure 24 arranged in a circle around the inner area 9 and protruding from the distal surface.

According to the illustrative embodiment of a planar part 8 shown in FIG. 8, ring-like shaped elements 25 protrude radially from the outer edge 11, while shaped elements 26 in the form of eyelets protrude from the surface of the planar part 8. These shaped elements can be made of titanium wire, for example.

FIG. 9 shows a plan view of a variant of the planar part 8 provided with through-openings 19′ which are formed as radial incisions from the outer edge 11 of the planar part 8 and are therefore radially open to the outside, the width of the incisions decreasing radially inward. The incisions are located exclusively in the outer area 10 of the planar part 8, so that the stiffness of the inner area 9 is not impaired by the through-openings 1′. The shape of the through-openings 19′ ensures an enhanced and outwardly increasing flexibility of the planar part 8 in the outer area 10.

As is illustrated schematically in FIG. 10, a common planar part 8 can be provided for several implant bodies 1, so that the planar part 8 radially surrounds all the implant bodies 1.

Conversely, according to FIG. 11, a single implant body 1 can also be connected to several planar parts 8 if the implant body 1, starting from the implant section 5, branches into several, in this case two, 1 skin penetration sections 6, which merge into a corresponding number of connector sections 7.

FIG. 12 illustrates how the distal surface, but also the proximal surface, of the planar part 8 can be divided into several zones 27, 28 and 29 in order to promote the adherence of different skin cells. Thus, the radial zone 27 can be designed especially for colonization with keratinocytes for formation of the epidermis.

By contrast, the zones 28 and 29 are provided for colonization with fibroblasts for formation of the connective tissue.

The surfaces of the planar parts 8, in particular the distal surfaces, can be configured with different surface structures so as to promote the adherence or incorporation of skin cells. These include microscopic surface structures, for example surface roughnesses, usually of 300 lim. Chemical or physical surface treatments can be used to modify the surface energy or surface charge, or wettability, and to functionalize the surface. Moreover, the surface can be provided with substances that promote the adherence of tissue. Such substances are, for example, collagens, growth factors, hormones or other proteins.

FIG. 13 illustrates how the planar part 8 can also be made up of mesh-like structures 30. In this embodiment, in order to form the stiff inner area 9 that represents an inherently non-moveable surface, an annular stiffening insert 31 is provided which has a greater material thickness in the inner area 9 than in the transition area 9′. The insert 31 does not extend into the outer area 10. In this way, the desired stiffness is obtained in the inner area 9. In the transition area 9′, the stiffness decreases, and the elasticity of the planar part 8 consequently increases. In the outer area 10, the elasticity of the planar part 8 is determined only by the elasticity of the mesh-like structure 30 within the material thickness.

A more or less similar variant of the planar part 8 is shown in FIG. 14, where a stiff insert 32 ensures that the enhanced stiffness already afforded by the material thickening in the inner area 9 is increased still further in the axial direction, as a result of which the attainable sealing effect is improved. Here, the insert 32 forms a part of the connection surface to the skin penetration section 6 and extends only partially into the inner area 9 of the planar part 8.

The embodiment shown in FIG. 15 illustrates that the implant body does not have to be made in one piece, and instead can have, for example, an implant section 5 into which a threaded

1-23. (canceled)

24. An implant with an implant body (1), comprising an implant section (5) intended to remain in the body, a skin penetration section (6), and an extracorporeal connector section (7), and with a planar part (8) which surrounds the skin penetration section (6), is provided for adhering to skin tissue and has a surface forming a support for a skin layer (4) surrounding the skin penetration section (6), characterized in that the planar part (8) adjoins the skin penetration section (6) in a sterile manner and comprises, in one integral piece, an inner area (9) adjoining the skin penetration section (6), and an outer area (10), the inner area (9) being made so stiff that it forms an inherently fixed support surface for a skin layer (4) surrounding the skin penetration section (6), and the outer area (10) has an elasticity adapted to the elasticity of the surrounding tissue.

25. The implant of claim 24, characterized in that the implant body (1) is a load-bearing part whose implant section (5) is designed as an anchoring section, and whose connector section (6) is designed for attachment of a load, in particular of a prosthesis.

26. The implant of claim 24, characterized in that the elasticity of the planar part (8) increases continuously toward the outside.

27. The implant of claim 24, characterized in that the planar part (8) is made of silicone.

28. The implant of claim 24, characterized in that the planar part (8) is formed from several individual pieces.

29. The implant of claim 24, characterized in that the planar part (8) has a multiplicity of through-openings (19, 19′).

30. The implant of claim 24, characterized in that at least one surface of the plate-like part (8) is provided with groove-like depressions (20, 21) or with webs.

31. The implant of claim 24, characterized in that shaped elements (23, 24, 25, 26) promoting the anchoring of tissue are applied at least on one part of at least one surface of the planar part (8).

32. The implant of claim 24, characterized in that the surface of the planar part (8) has, at least in some areas, a porous, net-like or roughened structure.

33. The implant of claim 24, characterized in that the surface is provided with substances that promote the adherence of tissue.

34. The implant of claim 24, characterized in that the planar part (8) has a substantially circular periphery.

35. The implant of claim 24, characterized in that the planar part (8) adjoins the implant body (1) substantially at right angles to a longitudinal axis of the implant body (1).

36. The implant of claim 24, characterized in that the planar part (8) is shaped like the top of a mushroom, with a jacket surface forming an acute angle with respect to a longitudinal axis of the implant body (1).

37. The implant of claim 24, characterized in that the planar part (8) is coated with bioactive material at least on one part of its surface.

38. The implant of claim 37, characterized in that the surface is coated in an island pattern with interstices.

39. The implant of claim 24, characterized in that the planar part (8) is connected releasably to the implant body (1).

40. The implant of claim 24, characterized in that, in order to receive the planar part (8), the implant body (1) has a stepped diameter reduction forming an annular shoulder (12).

41. The implant of claim 40, characterized in that the sterile connection is produced by laying the planar part (8) flat on the annular shoulder (12) of the implant body (1) with axial pretensioning.

42. The implant of claim 24, characterized in that the sterile connection is produced by a peripheral adhesive or weld filling the gap between the implant body (1) and the planar part (8).

43. The implant of claim 24, characterized in that the implant body (1) is provided with a bioactive surface at least in the area of connection to the distal surface of the planar part (8).

44. The implant of claim 24, characterized in that the implant body is provided, in its connector section (7), with a surface that prevents microorganisms settling on it.

45. The implant of claim 24, characterized in that several implant bodies (1) are connected to a common planar part (8).

46. The implant of claim 24, characterized in that, starting from an implant section (5), the implant body (1) branches into several skin penetration sections (6) which are connected to planar parts (8).
